(19) United States
(12) Patent Application Publication
(10) Pub. No.: US 2013/0031963 A1
(43) Pub. Date: Feb. 7, 2013

(54) WATER IN FUEL SENSOR

(71) Applicants: James A. Ritchie, JR., Huntertown, IN (US); Anthony W. Griffith, Cromwell, IN (US)

(72) Inventors: James A. Ritchie, JR., Huntertown, IN (US); Anthony W. Griffith, Cromwell, IN (US)

(21) Appl. No.: 13/566,058

(22) Filed: Aug. 3, 2012

Related U.S. Application Data
(60) Provisional application No. 61/515,655, filed on Aug. 5, 2011.

(51) Int. Cl. G01N 33/18

(52) U.S. Cl. 73/61.43

(57) ABSTRACT

A fuel system including a fuel container containing fluid and a water sensor. The water sensor is in at least partial fluid contact with the fluid. The water sensor has a longitudinal axis that is substantially horizontal. The water sensor is configured to sense water in the fluid at a predetermined level in the fuel container. The water sensor is further configured to sense water at the predetermined level regardless of a rotational position of the water sensor about the longitudinal axis.

### CROSS REFERENCE TO RELATED APPLICATIONS

This is a non-provisional application based upon U.S. provisional patent application Ser. No. 61/515,655 entitled “WATER IN FUEL SENSOR”, filed Aug. 5, 2011, which is incorporated herein by reference.

### BACKGROUND OF THE INVENTION

1. Field of the Invention

The present invention relates to engine sensors, and, more particularly, to water in fuel sensors.

2. Description of the Related Art

In the automotive industry, liquid fuel, such as diesel fuel, is cleaned of contaminants using a fuel filter inside of a fuel filter canister. The fuel flows into the canister, through the filter, and then out of the canister. The canister can have a generally cylindrical body. If water enters the canister, the water settles below the fuel in the canister and thus in the bottom portion of the interior of the canister, and the depth of the water continues to increase in the canister to the extent that water continues to enter the canister. The presence of water in fuel used to power automobiles is undesirable.

An electronic water-in-fuel sensor in the automotive industry is known which detects water in fuel in the fuel filter canister when the water reaches a certain depth in the canister. This sensor is threadably secured to a bottom portion of the fuel filter canister, and a portion of the sensor extends into the interior of the fuel canister. This sensor includes a thermoplastic housing, which is overmolded by way of injection molding over two parallel electrically conductive pins. This sensor is shown in FIG. 1. The pins are electrically connected with a voltage source and the engine control unit (ECU). The ends of the pins extend into the bottom portion of the interior of the canister. When the depth of the water in the canister is such that the water contacts the ends of both pins, then electricity flows from one pin, through the water, and to the other pin; this electrical connection is detected by the engine control unit, and remedial measures can then be taken. When the sensor is placed horizontally in the fuel filter canister, a level orientation of the two pins is required to ensure that the sensor detects the water level at the lowest level possible for the sensor. By contrast, if the pins were oriented vertically in the fuel filter canister, then the water would have to rise to the level of the highest pin before a signal would be sent to the engine control unit signifying the presence of water in the fuel filter canister.

What is needed in the art is an efficient water-in-fuel sensor that detects the presence of water in the fuel filter canister at a lower and consistent level.

### SUMMARY OF THE INVENTION

The present invention provides an efficient water-in-fuel sensor that detects the presence of water in the fuel filter canister at a lower level.

The present invention in one form is directed to a fuel system including a fuel container containing fluid and a water sensor. The water sensor is in at least partial fluid contact with the fluid. The water sensor has a longitudinal axis that is substantially horizontal. The water sensor is configured to sense water in the fluid at a predetermined level in the fuel container. The water sensor is further configured to sense water at the predetermined level regardless of a rotational position of the water sensor about the longitudinal axis.

The present invention in yet another form is directed to a method of using an electronic water-in-fuel sensor for detecting water in fuel in a fuel filter canister. The method includes the following steps: providing a sensor which includes a thermoplastic housing, a first pin, a second pin, a first dielectric element, a second dielectric element, a first disk, and a second disk, the first pin directly mechanically connected to and thereby directly electrically connected to the first disk, the second pin directly mechanically connected to and thereby directly electrically connected to the second disk, the first pin indirectly mechanically connected to the second disk by way of the first dielectric element, the second pin indirectly mechanically connected to the first disk by way of the second dielectric element, the thermoplastic housing at least partially encapsulating and mechanically interlocking with each of the first pin, the second pin, the first dielectric element, the second dielectric element, the first disk, and the second disk, the first disk electrically connecting with the second disk by way of water when water in the fuel filter canister contacts both the first and second disks at the same time; mounting the sensor to a bottom region of the fuel filter canister; sending an electric current to at least one of the first pin and the second pin; contacting both the first and second disks with water at the same time and thereby detecting the presence of water in the fuel filter canister; sending an electrical signal to a controller signifying the presence of water extending between the first and second disks.

An advantage of the present invention is that it provides 360 degree of water detection along the electrical contacts (the disks) and thereby eliminates thread-clocking issue of the water-in-fuel sensor of FIG. 1 (that is, that the pins of the sensor shown in FIG. 1 needs to be level to ensure the lowest possible water detection level for the sensor).

Another advantage is that it provides increased surface area of water-in-fuel contacts.

Yet another advantage is that it provides a lower detection point of water in fuel when placed horizontally in the fuel filter canister; stated another way, the electronic water-in-fuel sensor of the present invention provides a lower trigger level (triggering the presence of water) than the water-in-fuel sensor of FIG. 1.

Yet another advantage is that, by providing circular disks as the electrical contacts, a precise orientation of the disks is not required when screwing in the sensor relative to the fuel filter canister.

Yet another advantage of the present invention is that the electronic water-in-fuel sensor can be molded in one operation.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a side view of a prior art electronic water-in-fuel sensor;

FIG. 2 is a partially sectioned exploded side view of a fuel system showing the position of an embodiment of a water in fuel sensor of the present invention in a fuel filter canister;

FIG. 3 is a partially sectioned side view of the water sensor of FIG. 2;

FIG. 4 is a perspective view of the water sensor of FIGS. 2 and 3;

FIG. 5 is a perspective exploded view of the water sensor of FIGS. 2-4;

FIG. 6 is a perspective view of some of the contents of the water sensor of FIGS. 2-5;

FIG. 7 is a schematical representation of one embodiment of the circuit of the water sensor of FIGS. 2-6;

FIG. 8 is a schematical representation of another embodiment of the circuit of the water sensor of FIGS. 2-6;

FIG. 9 is a perspective view of an embodiment of a water sensor using the circuit depicted in FIG. 8;

FIG. 10 is an exploded perspective view of components of the water sensor of FIG. 9; and

FIG. 11 is a schematic representation of an embodiment of an instrumentation circuit using the water sensor of FIGS. 2-6.

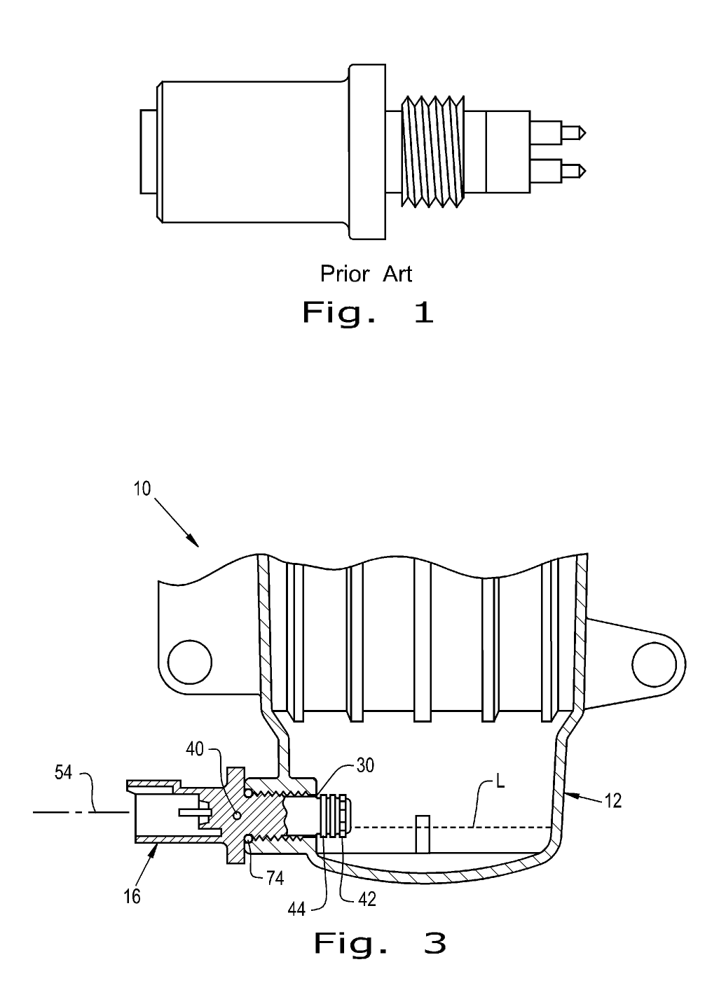

Corresponding reference characters indicate corresponding parts throughout the several views. The exemplifications set out herein illustrate embodiments of the invention, and such exemplifications are not to be construed as limiting the scope of the invention in any manner.

### DETAILED DESCRIPTION OF THE INVENTION

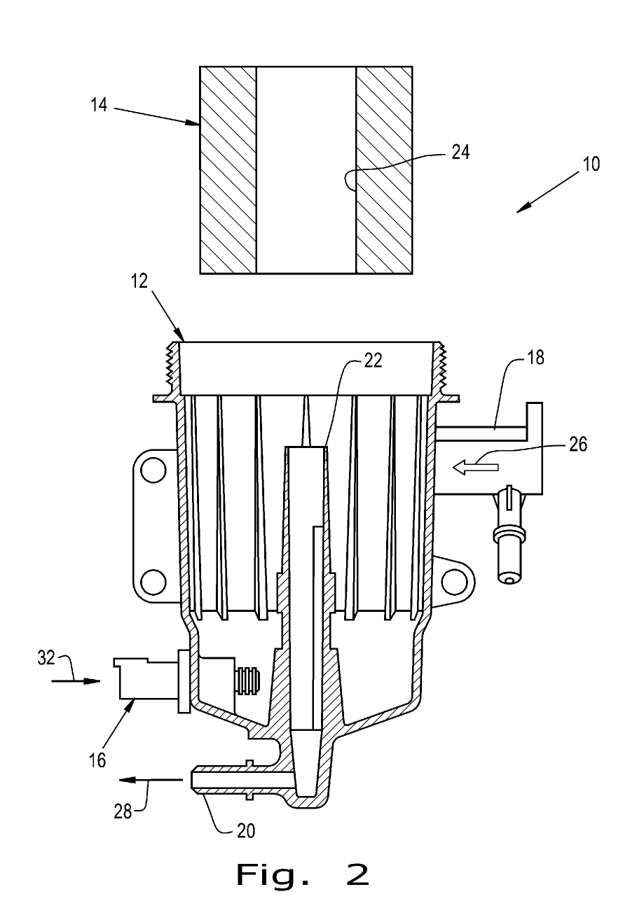

Referring now to the drawings, and more particularly to FIGS. 2 and 3, there is shown a fuel system 10 and more particularly a fuel filter system 10 which includes a fuel filter canister 12, a fuel filter 14 received by canister 12, and an electronic water-in-fuel sensor 16 which is mechanically connected to canister 12. Water sensor 16 can, for example and not by way of limitation, be removably, and thus replaceably, connected to canister 12 (and thus not be connected by way of welding); such a mechanical connection can be established by way of example, and not by way of limitation, a threadable connection (as shown in the drawings where sensor is threadably received by canister 12), a snap connection, a snap with clip connection, a latch connection, and/or a fastened connection. System 10 can be used for automobile applications, but is not necessarily limited to automobile applications, and is generally used for a liquid fuel system supplying fuel to a combustion engine. Canister 12 can be made of a polymeric material (for example and not by way of limitation, nylon) and includes a fuel input 18, a fuel output 20, and an upstanding tube 22 in the interior of canister 12 which leads to output 20. Fuel filter 14 is schematically shown and is inserted into canister 12. Fuel filter 14 generally speaking forms a passageway 24, which receives the upstanding tube 22 of canister 12. Fuel enters the interior of canister 12 by way of fuel input 18 (see arrow 26 showing the flow direction of the fuel into canister 12). The flow of the fuel, due to the volume of canister 12, has a reduced velocity while in canister 12 thereby allowing time for water to settle in the lower part of canister 12, perhaps accumulating to water level L. The fuel flows through fuel filter 14, then flows down the upstanding tube 22, and then flows out of fuel output 20 (see arrow 28 showing the flow direction of the fuel out of canister 12). By way of example and not by way of limitation, the fuel can be diesel fuel. The presence of water in the fuel canister 12 is undesirable; for example, water can foul fuel filter 14. But, water can become present in the fuel canister 12, such as by way of the fuel input 18 or possibly by condensation. Since water is denser than diesel fuel, water will settle below the fuel in the bottom interior of the fuel canister 12. Sensor 16 is configured for detecting the presence of water in the bottom of fuel filter canister 12. As the depth of the water rises in the bottom of canister 12, the water eventually contacts sensor 16 and can thereby be detected by sensor 16. Sensor 16 can be inserted, as a single unit, into a hole 30 of canister 12 (in the direction of arrow 32) and threadably connected thereto. Sensor 16 is thus an electrical water-in-fuel sensor 16, which is configured for detecting the presence of water in a fuel filter canister 12.

Sensor 16 is configured to detect water once it reaches a predetermined level, such as a predetermined level L, regardless of rotational orientation about longitudinal axis 54.

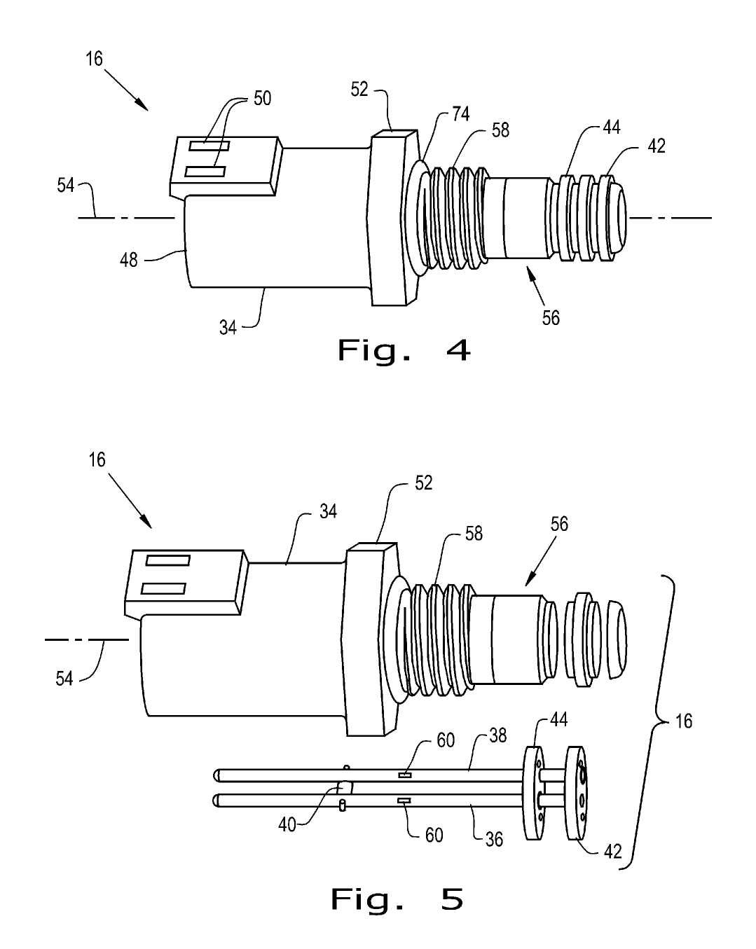

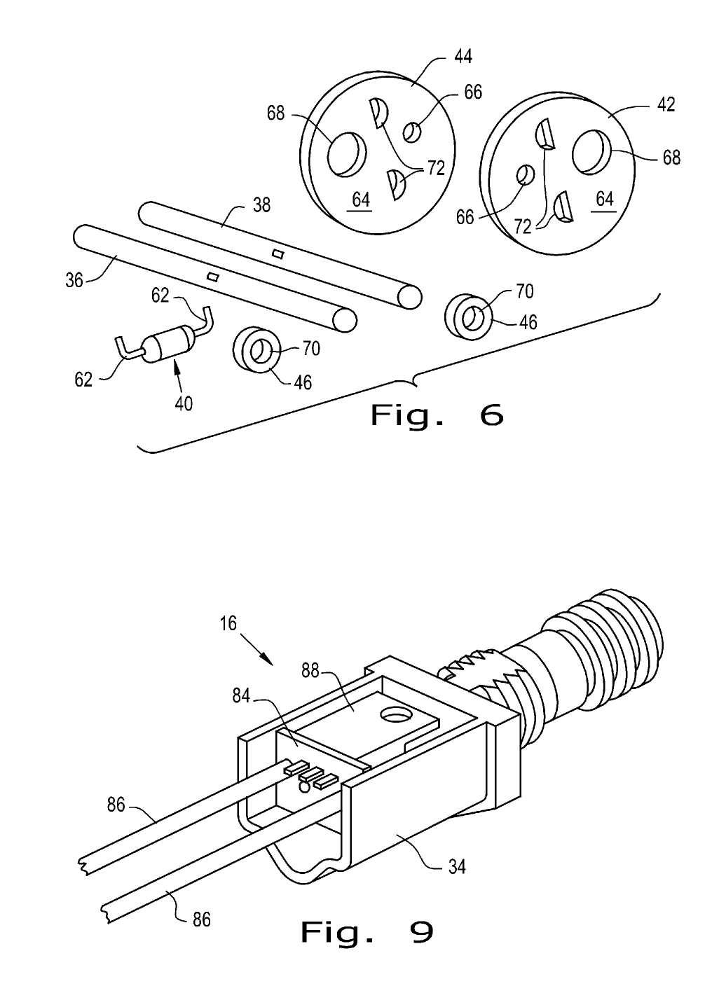

Now, additionally referring to FIGS. 4-11, and more particularly to FIGS. 4-6 sensor 16 is shown in more detail. Water sensor 16 includes a thermoplastic housing 34, a first pin 36, a second pin 38, a resistor 40, a first disk 42, a second disk 44, and dielectric inserts 46. By way of example and not by way of limitation, housing 34 can be made of a thermoplastic material such as nylon. The nylon material can have different compositions, such as mineral reinforcement or glass reinforcement. DuPont Minlon, for example, can be used as the material of housing 34. Housing 34 can be formed by injection molding, and, in so doing, housing 34 can overmold and thereby encapsulate pins 36 and 38, resistor 40, and disks 42 and 44. Housing 34 shown in the drawings includes a proximal end which forms a socket 48 for receiving an electrical connector, which can be connected to electrical conductors running to an engine control unit 78 (for example, the proximal end of the sensor 16 is to the left in FIGS. 4 and 5, and the distal end of the sensor 16 is to the right, the distal end including disks 42 and 44). Housing 34 has two holes 50 in the housing above socket 48, holes 50 being used to receive a latch mechanism of an electrical connector which includes terminals to mate with pins 36 and 38 in socket 48, the latch mechanism being used to secure such an electrical connector to socket 48 (for example, and not by way of limitation, this electrical connector can be a Delphi connector, a Delphi-Packard connector, or a Deutsch connector). This electrical connector (not shown) can include wires which extend to an automotive controller such as an engine control unit. Housing 34 includes a wall 52, which is generally perpendicular to longitudinal axis 54 of sensor 16, this wall 52 forming an abutment against canister 12. Housing 34 further includes a projecting arm 56, which projects from the wall 52. The projecting arm 56 includes threads 58, which threadably engage threads of a sensor insertion hole 30, formed by canister 12. At least a portion of the projecting arm 56 extends into the interior of canister 12.

Pins 36 and 38 are electrical conductors and can be substantially identical to one another. Pins 36 and 38 extend longitudinally, generally parallel to one another, and generally parallel to longitudinal axis 54 of sensor 16. Pins 36 and 38 can be formed, for example and not by way of limitation, by stamping or lathe machining. Each pin 36 and 38 can include at least one interlock feature 60, such as a female divot 60, which mechanically interlocks with housing 34 by way of overmolding. Each pin 36 and 38 can include four such divots 60 spaced circumferentially about the respective pin 36 and 38, each divot 60 receiving housing material during an overmolding operation; these divots 60 can be formed by stamping or machining (such as laser machining). More or less divots 60 can be formed in pins 36 and 38. Other mechanical interlocking ways can be formed on pins 36 and 38 in addition to or instead of divots 60; for example, a channel extending 360 degrees around each pin can be turned (i.e., turned by lathe machining) into the circumference of each pin 36 and 38, each channel receiving housing material during molding. By way of example and not by way of limitation, each pin 36 and 38 can be made of an electrically conductive metal such as stainless steel (for example, 304 stainless steel), non-ferrous metals (for example, brass, nickel, or others), copper-based materials, or conductive alloys. In some applications, pins 36 and 38 made of 304 stainless steel can be nickel plated (i.e., for soldering pins 36 and 38 to a printed circuit board). After overmolding the housing 34 over pins 36 and 38, pins 36 and 38 can be visible and accessible by way of socket 48 of housing 34. Pins 36 and 38 are coupled with disks 42 and 44, as described below.

FIG. 5, for example, shows a resistor 40 extending between pins 36 and 38. Resistor 40 can be overmolded by housing 34. Resistor 40 can include electrically conductive lines 62 which can be attached respectively to pins 36 and 38 by way of, for example (and not by way of limitation), ultrasonic welding. Resistor 40 forms one electrical path for electrically connecting pins 36 and 38 together. Resistor 40 is optional in sensor 16; stated another way, sensor 16 can be formed without resistor 40. Resistor 40 can have an electrical resistance value, for example, of 82 KΩ (other values, such as for example 105 KΩ or 205 KΩ or other values, can be used instead, depending upon the application).

Each disk 42 and 44 is electrically conductive and has a circular cross-section. The diameter of each disk 42 and 44 is substantially identical relative to one another. Longitudinal axis 54 of sensor 16 can proceed through the center of each disk 42 and 44. Second disk 44 is positioned rearward (towards wall 50) of first disk 42. Each disk 42 and 44 can also be referred to as a washer, a washer contact, a water-in-fuel contact, a contact, a water-in-fuel contact ring, or an electrical contact or conductor. By way of example and not by way of limitation, each disk 42 and 44 can be made of an electrically conductive metal such as stainless steel (for example, 304 stainless steel), non-ferrous metals (for example, brass, nickel, or others), copper-based materials, or conductive alloys. Each disk 42 and 44 can be substantially identical to one another (as shown in the drawings); however, second disk 44 is turned around relative to first disk 42 when disks 42 and 44 are attached to pins 36 and 38. Each disk 42 and 44 can include opposing flat faces 64 which are substantially parallel to one another. Further, while disks 42 and 44 are spaced apart from one another, each disk 42 and 44 can be positioned substantially parallel to one another. The spacing between disks 42 and 44 can be in-filled by the material of housing 34. Further, as shown in the drawings, disks 42 and 44 can be formed integral with housing 34 and pins 36 and 38 such that disks 42 and 44 are not intended to be detached from housing 34 or pins 36 and 38 during normal operation of sensor 16. A circumferential region of each disk 42 and 44 extends beyond housing 34.

First disk 42 includes a first through-hole 66 and a second through-hole 68. First hole 66 is connected with the distal end of first pin 36 by way of, for example, a press fit, the distal end of first pin 36 generally being flush with a distal face 64 of first disk 42. First disk 42 is thus directly connected (both mechanically and electrically) to first pin 36 by way of first hole 66. Second hole 68 can have a larger diameter than first hole 66. Second hole 68 receives a respective dielectric insert 46 formed of an insulative material (insert 46 can also be referred to as a spacer). By way of example and not by way of limitation, dielectric insert 46 can be made of a plastic material such as nylon and can be formed by way of a molding operation, such as injection molding. The dielectric insert 46 is inserted into second hole 68 and includes a through-hole 70, the dielectric insert 46 being connected to second hole 68 by way of a press fit, as one option. Through-hole 70 of dielectric insert 46 receives the distal end of second pin 38 and can be connected to pin 38, for example and not by way of limitation, using a press fit. Dielectric insert 46 electrically insulates (and thereby electrically isolates) first disk 42 from second pin 38; dielectric insert 46 provides a clearance between first disk 42 and second pin 38. First disk 42 further includes two through-holes 72 for receiving the material of housing 34 during injection molding; that is the material forming housing 34 flows into and through through-holes 72 during injection molding and thereby mechanically interlocks housing 34 with first disk 36.

Second disk 44 is spaced apart from first disk 42 but is otherwise positioned on the distal end regions of first and second pins 36 and 38. Second disk 44 includes a first through-hole 66 and a second through-hole 68. First hole 66 is connected with second pin 38 by way of, for example, a press fit, second pin 38 extending all of the way through first hole 66 of second disk 44 and into through-hole 70 of dielectric insert 46. Second disk 44 is thus directly connected to second pin 38 by way of first hole 66. Second hole 68 can have a larger diameter than first hole 66. Second hole 68 receives a respective dielectric insert 46 formed of an insulative material. The dielectric insert 46 associated with second disk 44 is substantially similar to dielectric insert 46 of first disk 42. The dielectric insert 46 is inserted into the second hole 68 and includes a through-hole 70, the dielectric insert 46 being connected to second hole 68 by way of a press fit, as one option. Through-hole 70 of dielectric insert 46 receives therethrough first pin 36 and can be connected to pin 36, for example and not by way of limitation, using a press fit. Dielectric insert 46 electrically insulates (and thereby electrically isolates) second disk 44 from first pin 36; dielectric insert 46 provides a clearance between second disk 44 and first pin 36. Second disk 44 further includes two through-holes 72 for receiving the material of housing 34 during injection molding; that is the material forming housing 34 flows into and through through-holes 72 during injection molding and thereby mechanically interlocks housing 34 with second disk 44.

According to an alternative embodiment of the present invention, dielectric inserts 46 can be omitted from disks 42 and 44. The material of housing 34, instead, can be substituted for dielectric inserts 46. More specifically, during the molding operation forming housing 34, the material of housing 34 can flow into the clearance between first pin 36 and second disk 44 and also into the clearance between second pin 38 and first disk 42 and thereby insulate first pin 36 from second disk 44 and second pin 38 from first disk 42 at these coupling points.

Disks 42 and 44 (including holes 66, 68, and 72) can be formed, for example, by a machining operation, such as laser machining or stamping. Dielectric inserts 46 can be press fit into position respectively on pins 36 and 38, and then dielectric inserts 46 (along with pins) can be placed into position using a press fit into the respective hole 68 of disks 42 and 44. Alternatively, dielectric inserts 46 can be placed into position using a press fit into the respective holes 68 of disks 42 and 44, and then pins 36 and 38 can be press fit into position respectively within respective holes 70 of dielectric inserts 46. This assembly can be placed in a fixture so that resistor 40 can be welded to pins 36 and 38. This assembly including resistor 40 can then be placed into mold tooling and housing 34 can be overmolded over the electronic components 36, 38, 40, 42 and 44, and dielectric inserts 46. If dielectric inserts 46 are omitted, pins 36 and 38 and disks 42 and 44 can be placed in a fixture so as to maintain a clearance gap in holes 68; after welding resistor 40 onto pins 36 and 38, this assembly can then be placed into mold tooling to overmold housing 34 over the electronic components 36, 38, 40, 42 and 44.

A seal 74, such as an 0-ring seal 74, is placed on housing 34 of sensor 16. By way of example and not by way of limitation, seal 74 can be made of Viton (which is understood to be a brand of synthetic rubber and fluoropolymer elastomer). Seal 74 helps to seal any clearance gap between sensor 16 and canister 12.

Sensor 16 is threadably secured to fuel filter canister 12, which may be associated with an automobile for example. An electric current at a predetermined voltage (which can be varying) flows to sensor 16. For example and not by way of limitation, the voltage can normally be at approximately 12 volts but can range between 6 and 28 volts or any other voltage. Depending upon the application, the current supplied to sensor can be direct current (constant or pulsating direct current) or alternating current. The power source can be any power source depending upon the application (i.e., a battery, an alternator, solar cells). The voltage signal to sensor 16 can come from engine control unit 78 (for example, by way of capacitors, batteries, or other electronic storage devices within the engine control unit, which can also be referred to as an engine control module), depending upon the application, or can come to sensor 16 not by way of the engine control unit (for example, the voltage source could be in series with the engine control unit). Electric current flows through resistor 40, by way of pins 36 and 38 and to the engine control unit of the automobile. The resistance of resistor 40 has a predetermined value. Since first pin 36 is directly connected to first disk 42 but insulated relative to second disk 44 by way of dielectric insert 46 in second hole 68 of second disk 44, and since second pin 38 is directly connected to second disk 44 but insulated relative to first disk 42 by way of dielectric insert 46 in second hole 68 of first disk 42, first pin 36 and first disk 42 are directly electrically connected to one another, second pin 38 and second disk 44 are directly electrically connected to one another, but no electrical current flows between first and second disks 42 and 44; in this sense, disks 42 and 44 are electrically isolated from one another (absent the presence of a conductive fluid such as water). However, when water in the fuel filter canister 12 touches both disks 42 and 44 at the same time, then an electrical connection is made between the disks 42 and 44 by way of the water.

More specifically, an electric current flows through first disk 42, the water, and second disk 44. The flow of current through this path alters the equivalent resistance of water sensor 16, and this is detected by the change in voltage and/or current characteristics by engine control unit 78. The electric current path through the resistor 40 is parallel with the electric current path through the water. When the electric current path is formed through the water, an electrical signal is sent to the engine control unit by way of second pin 38, and the engine control unit can detect a change of resistance in the circuit associated with sensor 16. For instance, with a resistance of 82 KΩ the engine control unit looks for a resistance of 82 KΩ from the sensor 16 within a tolerance level; when the resistance from the sensor 16 changes outside the tolerance, then the engine control unit concludes that a current path has been formed between disks 42 and 44 by way of the water. The engine control unit can detect this change in resistance by measuring the voltage of the return signal, which is expected to change at the engine control unit with a change in resistance in the circuit (due to the presence of the water). More specifically, the engine control unit can detect the reduced overall resistance provided by the water by measuring the voltage in the return signal from sensor 16 and determining whether this voltage signal falls within a predetermined percentage of the voltage supply. If the voltage signal is within a predetermined percentage of the voltage supply, then the engine control unit can determine that only fuel (i.e., diesel) is present; if the voltage signal is outside this predetermined percentage of the voltage supply (that is, a voltage which is lower than the acceptable range for the fuel and possibly within another predetermined range that is associated with water), then the engine control unit determines that water is present and contacting sensor disks 42 and 44. The presence of water in the fuel filter canister 12 can then be detected. For example, a water level in the bottom of the canister 12, which has risen above the bottom of disks 42 and 44, thereby provides an electrically conductive current flow path between disks 42 and 44. Upon detecting the water, the engine control unit can take remedial action to alert an operator of the automobile by way of an indicator light 92, or by an alarm, or by other means.

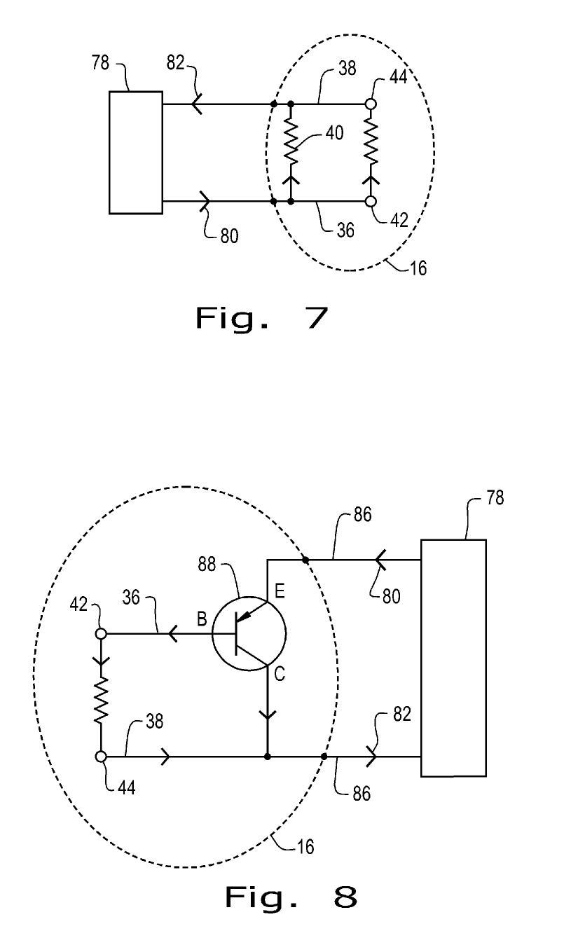

A schematic of an example of an electric circuit diagram is provided at FIG. 7 (this electric circuit diagram can apply to the preceding paragraph herein). Controller 78, such as an engine control unit 78, and sensor 16 including resistor 40 and disks 42 and 44. The resistor symbol between disks 42 and 44 represents the current flow path through the water when water is present; it is understood that the resistance value of the water can vary. The input signal, which can be understood to be a voltage level applied to sensor 16 causes a current flow that is represented by arrowheads 80 and 82. The resistance value between disks 42 and 44 is associated with water and if no water is present the resistance value may be infinite, so that the voltage applied to water sensor 16 is at a predetermined value and the current flow is as determined by the current flowing through resistor 40. When water is contacting disks 42 and 44 the resistance value between disks 42 and 44 is significantly less than the effective open circuit that exists when only diesel fuel is present in container 12. This causes a larger current flow in water sensor 16 and perhaps a lower voltage due the increased load caused by the reduced resistance, which is in turn detected by engine control unit 78.

According to an alternative embodiment of sensor 16 according to the present invention, resistor 40 may be omitted (this embodiment of the present invention is not separately shown, but can be understood to have the circuit equivalent to that shown in FIG. 7, with resistor 40 omitted). In such a case, pins 36 and 38 and disks 42 and 44 can be electrically isolated from one another until water closes the circuit by electrically connecting disks 42 and 44 together. That is, an open circuit exists between pins 36 and 38 without the presence of water. The electrical connection made between disks 42 and 44 (and thus also pins 36 and 38) through the water can be detected by the engine control unit as described above. The sensor 16 in this embodiment is understood to be an analog (passive) device.

According to an alternative embodiment of the present invention, the connection between sensor 16 and electrical lines running to the engine control unit could be made differently. Rather than having a removable electrical connector which connects to pins 36 and 38 by way of socket 48, respective electrical terminals can be mechanically and electrically connected to pins 36 and 38, and these pins can then be secured to socket 48 by way of epoxy (socket 48 can be formed as shown in the drawings or could be formed differently but having a pocket for receiving and holding the epoxy). In this way the terminals (which form a part of an electrical harness running, for example, to the engine control unit) can be hardwired to the sensor according to the present invention.

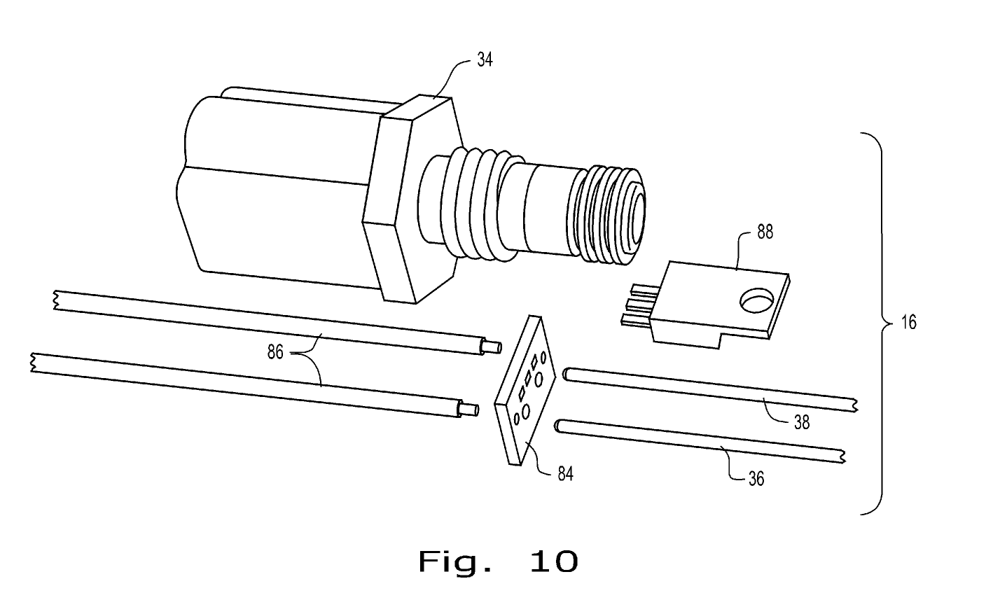

Now paying particular attention to FIGS. 8-10, there is shown another alternative embodiment of the present invention, where resistor 40 is omitted and a transistor 88 is used in place of resistor 40. A schematic illustration of an example of a circuit diagram including transistor 88 is shown in FIG. 8. Pins 36 and 38 are connected to a printed circuit board (PCB) 84, and wires 86 are also connected to the PCB 84, with wires 86 running to engine control unit 78. The transistor 88 is mounted to PCB 84. Socket 48 can be filled with epoxy to secure PCB 84 to housing 34 (epoxy can fill the rear end of socket 48 to the rear of PCB 84 or can fill socket 48 in front of and to the rear of PCB 84). For example and not by way of limitation, pins 36 and 38 can be 304 stainless steel and nickel-plated. Pins 36 and 38 can be soldered to PCB 84. Wire leads 86 are electrically conductive and can be soldered to PCB 84. The three terminals of transistor 88 can also be soldered to PCB 84. By way of example and not by way of limitation, the following connections can be made. Wire 86 running from engine control unit 78 to sensor 16 can be electrically connected to a terminal of transistor 88 shown in FIG. 8 as emitter E. Further, by way of PCB 84, pin 36 can be connected to another terminal of transistor shown to be base B, and pin 36 can be connected to disk 42. Further, by way of PCB 84, pin 38 can be electrically connected to another terminal of the transistor 88 collector C and to one of wires 86 running to engine control unit 78, and pin 38 can be connected to disk 44. While a PNP bipolar junction transistor is shown in FIG. 8, it is understood that the circuit associated with sensor 16 could include, rather, an NPN bipolar junction transistor. As described above, an open circuit between disks 42 and 44 exists until water contacts both disks 42 and 44 at the same time, which is schematically shown as a resistor between disks 42 and 44. Except for some leakage current no current passes through transistor 88 unless water is present between disks 42 and 44. It is understood that the voltage source for this embodiment can be as described above.

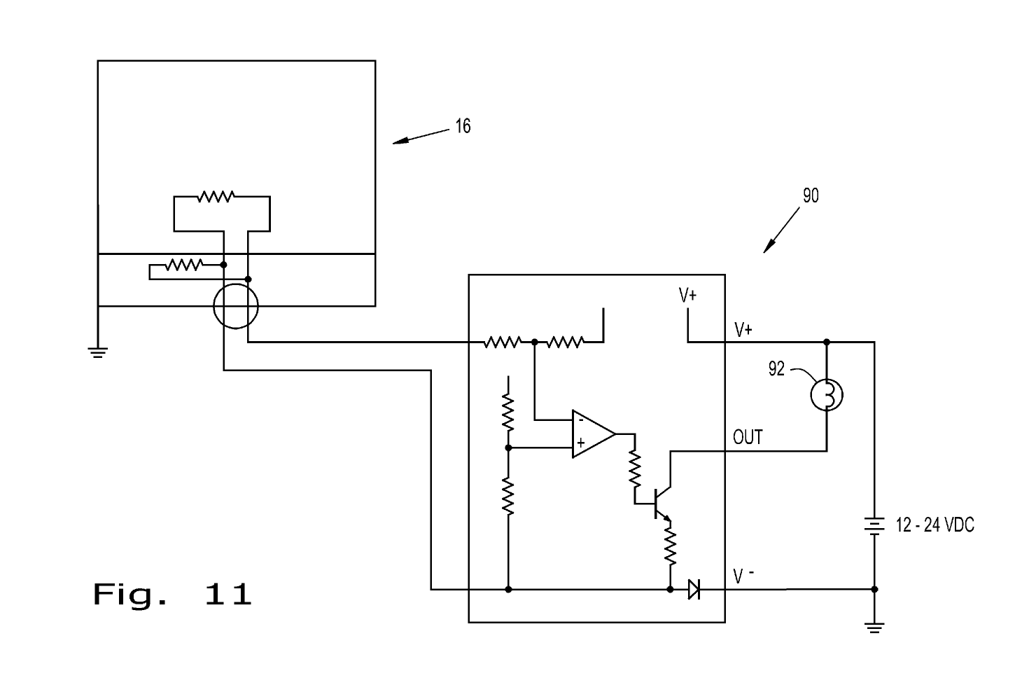

According to an alternative embodiment of the present invention, the sensor can be formed as a digital water-in-fuel (WIF) sensor. The digital WIF sensor includes a PCB and other electrical components onboard the sensor itself to signal water in diesel fuel. The PCB is mated into the rear socket of the sensor. In this variation, at least a portion of the controlling functions of the engine control unit can be contained in sensor 16. That is, the electrical components on the PCB receive the signal from the disks that water is present and can determine that water is indeed present. It is understood that this embodiment can employ pins, disks, dielectric inserts and be connected to each other and the sensor housing as described above (with or without the dielectric inserts). The PCB can be attached to the back of the sensor housing by way of filling the socket with epoxy. Electrical conductors and/or an electrical harness can be attached to the terminals extending from the back of the sensor (shown in FIG. 10) and run, directly or indirectly and individually or together, to a voltage source and/or a warning indicator and/or another controller, for example.

The present invention in one form is directed to an electronic water-in-fuel sensor 16 for detecting water in fuel in a fuel filter canister 12. The sensor 16 includes a thermoplastic housing 34, a first pin 36, a second pin 38, a first dielectric element 46, a second dielectric element 46, a first disk 42, and a second disk 44, the first pin 36 directly mechanically connected to and thereby directly electrically connected to the first disk 42, the second pin 38 directly mechanically connected to and thereby directly electrically connected to the second disk 44, the first pin 36 indirectly mechanically connected to the second disk 44 by way of the first dielectric element 46, the second pin 36 indirectly mechanically connected to the first disk 42 by way of the second dielectric element 46, the thermoplastic housing 34 at least partially encapsulating and mechanically interlocking with each of the first pin 36, the second pin 38, the first dielectric element 46, the second dielectric element 46, the first disk 42, and the second disk 44, the first disk 42 electrically connecting with the second disk 44 by way of water when water in the fuel filter canister 12 contacts both the first and second disks 42 and 44 at the same time.

The present invention in yet another form is directed to a method of using an electronic water-in-fuel sensor 16 for detecting water in fuel in a fuel filter canister 12. The method includes the following steps: providing a sensor 16 which includes a thermoplastic housing 34, a first pin 36, a second pin 38, a first dielectric element 46, a second dielectric element 46, a first disk 42, and a second disk 44, the first pin 36 directly mechanically connected to and thereby directly electrically connected to the first disk 42, the second pin 38 directly mechanically connected to and thereby directly electrically connected to the second disk 44, the first pin 36 indirectly mechanically connected to the second disk 44 by way of the first dielectric element 46, the second pin 38 indirectly mechanically connected to the first disk 42 by way of the second dielectric element 46, the thermoplastic housing 34 at least partially encapsulating and mechanically interlocking with each of the first pin 36, the second pin 38, the first dielectric element 46, the second dielectric element 46, the first disk 42, and the second disk 44, the first disk 42 electrically connecting with the second disk 44 by way of water when water in the fuel filter canister 12 contacts both the first and second disks 42 and 44 at the same time; mounting the sensor 16 to a bottom region of the fuel filter canister 12; sending an electric current to at least one of the first pin 36 and the second pin 38; contacting both the first and second disks 42 and 44 with water at the same time and thereby detecting the presence of water in the fuel filter canister 12; sending an electrical signal to a controller signifying the presence of water extending between the first and second disks 42 and 44.

The schematic of FIG. 11 illustrates a monitoring circuit 90, which may be in ECU 78 that is electrically connected to sensor 16. When the presence of water in the fluid is detected by a change in resistance between disks 42 and 44, which can be considered annular electrical conductors, and is so determined by monitoring circuit 90 that uses an operational amplifier to detect the change in current flow, an indicator 92 is illuminated to alert an operator of the engine. Indicator 92 is illustrated as a light, but it is understood that it can also generate an error signal or message in some other known fashion.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A fuel system, comprising:
a fuel container containing fluid; and
a water sensor in at least partial fluid contact with the fluid, said water sensor having a longitudinal axis, said longitudinal axis being substantially horizontal, said water sensor being configured to sense water in the fluid at a predetermined level in said fuel container, said water sensor being further configured to sense water at the predetermined level regardless of a rotational position of said water sensor about said longitudinal axis.

2. The fuel system of claim 1, wherein said water sensor has at least two annular electrical conductors in contact with the fluid.

3. The fuel system of claim 2, wherein said at least two annular electrical conductors are substantially the same size.

4. The fuel system of claim 3, wherein said at least two annular electrical conductors are substantially parallel with each other.

5. The fuel system of claim 4, wherein said at least two annular electrical conductors are disk-like in shape.

6. The fuel system of claim 5, wherein said at least two annular electrical conductors are substantially normal to said longitudinal axis.

7. The fuel system of claim 6, wherein said water sensor additionally includes at least one other electrical conductor, said two annular electrical conductors including a first annular electrical conductor and a second annular electrical conductor, said first annular electrical conductor having a hole therethrough with said other electrical conductor passing through the hole, said other electrical conductor not being directly electrically connected to said first annular electrical conductor, said other electrical conductor being directly electrically connected to said second annular electrical conductor

8. The fuel system of claim 1, wherein said water sensor further includes a first electrical conductor and a second electrical conductor each circumscribing an outer surface of said water sensor, said at least two electrical conductors being in contact with the fluid.

9. The fuel system of claim 8, wherein said water sensor further includes a third electrical conductor and a fourth electrical conductor, said third electrical conductor being in direct electrical contact with said first electrical conductor, said fourth electrical conductor being in direct electrical contact with said second electrical conductor, said fourth electrical conductor passing longitudinally through said first electrical conductor.

10. The fuel system of claim 9, wherein said water sensor further includes an electrical component electrically connected between said third electrical conductor and said fourth electrical conductor.

11. A water sensor for use with a fuel container containing a fluid, the water sensor comprising:
a structural body having a longitudinal axis, said longitudinal axis being substantially horizontal, the water sensor being configured to detect water in the fluid at a predetermined level in the fuel container, the water sensor being further configured to detect water at the predetermined level regardless of a rotational orientation of said structural body about said longitudinal axis.

12. The water sensor of claim 11, further comprising at least two annular electrical conductors in contact with the fluid.

13. The water sensor of claim 12, wherein said at least two annular electrical conductors are substantially the same size.

14. The water sensor of claim 13, wherein said at least two annular electrical conductors are substantially parallel with each other.

15. The water sensor of claim 14, wherein said at least two annular electrical conductors are disk-like in shape.

16. The water sensor of claim 15, wherein said at least two annular electrical conductors are substantially normal to said longitudinal axis.

17. The water sensor of claim 16, further comprising at least one other electrical conductor, said two annular electrical conductors including a first annular electrical conductor and a second annular electrical conductor, said first annular electrical conductor having a hole therethrough with said other electrical conductor passing through the hole, said other electrical conductor not being directly electrically connected to said first annular electrical conductor, said other electrical conductor being directly electrically connected to said second annular electrical conductor

18. The water sensor of claim 11, further comprising a first electrical conductor and a second electrical conductor each circumscribing an outer surface of said structural body, said at least two electrical conductors being in contact with the fluid.

19. The water sensor of claim 18, further comprising:
a third electrical conductor; and
a fourth electrical conductor, said third electrical conductor being in direct electrical contact with said first electrical conductor, said fourth electrical conductor being in direct electrical contact with said second electrical conductor, said fourth electrical conductor passing longitudinally through said first electrical conductor.

20. The water sensor of claim 19, wherein said water sensor further includes an electrical component electrically connected between said third electrical conductor and said fourth electrical conductor.
